(19) United States
(12) Patent Application Publication
(10) Pub. No.: US 2008/0317826 A1
(43) Pub. Date: Dec. 25, 2008

(54) POROUS KERATIN CONSTRUCTS, WOUND HEALING ASSEMBLIES AND METHODS USING THE SAME

(76) Inventors: Robert James Kelly, Christchurch (NZ); Gudmunder Fertram Sigurjonsson, Auckland (NZ); Clive Marsh, Christchurch (NZ); Robert Allen Smith, Jackson, MS (US); Mohammad Azam Ali, Christchurch (NZ)

Correspondence Address: HOLLAND & HART, LLP, P.O BOX 8749, DENVER, CO, 80201, US

(21) Appl. No.: 12/127,694

(22) Filed: May 27, 2008

Related U.S. Application Data
(60) Provisional application No. 60/924,649, filed on May 24, 2007.

(51) Int. Cl. A61K 9/70; A61K 38/17; A61P 19/00

(52) U.S. Cl. 424/443; 514/12

(57) ABSTRACT

A porous keratin construct for use in wound healing is disclosed. The porous keratin construct may be used standing alone or in combination with a synthetic foam backing layer. Either the porous keratin construct or the porous keratin construct and synthetic foam combination may be used in a wound therapy such as negative pressure wound therapy. An assembly for use in negative pressure wound therapy may comprise a porous keratin construct or porous keratin construct and synthetic foam combination, a wound drape to encapsulate the wound and the porous keratin construct or porous keratin construct and synthetic foam combination, and a vacuum source in fluid communication with the wound drape to apply a negative pressure to the area encapsulated by the wound drape

This application claims priority to U.S. Provisional Application Ser. No. 60/924,032, filed May 24, 2007, the entirety of which is hereby incorporated by reference.

### FIELD

This disclosure relates generally to porous keratin constructs and their various uses in different methods of wound healing. More particularly, the present disclosure relates to a porous keratin construct to enhance wound healing and which may be used as, for example, a pad applied directly on a wound or as a spacer or interface used in vacuum induced healing of open wounds.

### BACKGROUND

Chronic wounds can be caused by a variety of events, including surgery, prolonged bed rest, and traumatic injuries. Partial thickness wounds can include second degree burns, abrasions, and skin graft donor sites. Healing of these wounds can be problematic, especially in cases of diabetes mellitus or chronic immune disorders. Full thickness wounds have no skin remaining, and can be the result of trauma, diabetes (e.g., leg ulcers), and venous stasis disease, which can cause full thickness ulcers of the lower extremities. Full thickness wounds tend to heal very slowly. Proper wound care technique, including the use of wound dressings, is extremely important to successful chronic wound management. Chronic wounds affect an estimated four million people a year, resulting in health care costs in the billions of dollars.

The wound healing process involves a complex series of biological interactions at the cellular level, which can be grouped into three phases: hemostasis and inflammation, granulation tissue formation and re-epithelization, and remodeling. Keratinocytes (epidermal cells that manufacture and contain keratin) migrate from wound edges to cover the wound. Growth factors such as transforming growth factor-β (TGF-β) play a critical role in stimulating the migration process. The migration occurs optimally under the cover of a moist layer.

Keratins have been found to be necessary for the re-epithelization phase of the wound healing process. Keratins are major structural proteins of all epithelial cell types and appear to play a major role in wound healing.

Although not ideal for chronic wounds, several wound dressings are currently on the market, including occlusive dressings, non-adherent wound dressings and dressings in the form of sheets, foams, powders and gels. However, these wound dressings are not optimal and face several problems. For example, many existing wound dressings fail to manage exudates while still providing a beneficial material (such as keratin) to wounds. Additionally, wound dressings comprising layers of protein on synthetic foam tend to prevent uptake of exudates because the protein layers tend to ingress into the foam. Finally, existing wound dressings do not prevent oxidative stress associated with highly exuding wounds. Accordingly, a wound dressing suitable to be placed directly into a wound that addresses some or all of these issues is desirable.

Additionally, certain severe wounds require treatment that goes beyond merely placing a wound dressing directly on to the wound in order to achieve effective healing. As is well known to those of ordinary skill in the art, closure of surface wounds involves the inward migration of epithelial and subcutaneous tissue adjacent the wound. This migration is ordinarily assisted through the inflammatory process, whereby blood flow is increased and various functional cell types are activated. Through the inflammatory process, blood flow through damaged or broken vessels is stopped by capillary level occlusion; thereafter, cleanup and rebuilding operations may begin. Unfortunately, this process is hampered when a wound is large or has become infected. In such wounds, a zone of stasis (i.e., an area in which localized swelling of tissue restricts the flow of blood to the tissues) forms near the surface of the wound.

Without sufficient blood flow, the epithelial and subcutaneous tissues surrounding the wound not only receive diminished oxygen and nutrients, but are also less able to successfully fight bacterial infection and thus are less able to naturally close the wound. In the past, such difficult wounds were addressed only through the use of sutures or staples. Although still widely practiced and often effective, such mechanical closure techniques suffer a major disadvantage in that they produce tension on the skin tissue adjacent the wound. In particular, the tensile force required in order to achieve closure using sutures or staples may cause very high localized stresses at the suture or staple insertion point. These stresses commonly result in the rupture of the tissue at the insertion points, which can eventually cause wound dehiscence and additional tissue loss.

Additionally, some wounds harden and inflame to such a degree due to infection that closure by stapling or suturing is not feasible. Wounds not reparable by suturing or stapling generally require prolonged hospitalization, with its attendant high cost, and major surgical procedures, such as grafts of surrounding tissues. Examples of wounds not readily treatable with staples or suturing include large, deep, open wounds; decubitus ulcers; ulcers resulting from chronic osteomyelitis; and partial thickness burns that subsequently develop into full thickness burns.

One such alternative method of treating these types of wounds is vacuum induced healing. Vacuum induced healing of open wounds has recently been popularized by Kinetic Concepts, Inc. of San Antonio, Tex., by its commercially available V.A.C.® product line. The vacuum induced healing process has been described in U.S. Pat. No. 4,969,880 issued on Nov. 13, 1990 to Zarnierowski, as well as its continuations and continuations in part, U.S. Pat. No. 5,100,396, issued on Mar. 31, 1992, U.S. Pat. No. 5,261,893, issued Nov. 16, 1993, and U.S. Pat. No. 5,527,293, issued Jun. 18, 1996, the disclosures of which are incorporated herein by this reference. Further improvements and modifications of the vacuum induced healing process are also described in U.S. Pat. No. 6,071,267, issued on Jun. 6, 2000 to Zamierowski and U.S. Pat. Nos. 5,636,643 and 5,645,081 issued to Argenta et al. on Jun. 10, 1997 and Jul. 8, 1997 respectively, the disclosures of which are incorporated by reference as though fully set forth herein.

As a result of the shortcomings of mechanical closure devices described above, methods and apparatus for draining wounds by applying continuous negative pressure have been developed. When applied over a sufficient area of the wound, such negative pressures have been found to promote the migration toward the wound of epithelial and subcutaneous tissues. In practice, the application to a wound of negative gauge pressure, commercialized by KCl Licensing, Inc., San Antonio, Tex., under the designation “Vacuum Assisted Closure” (or “V.A.C.®”) therapy, typically involves the mechanical-like contraction of the wound with simultaneous removal of excess fluid. In this manner, V.A.C.® therapy augments the body's natural inflammatory process while alleviating many of the known intrinsic side effects, such as the production of edema caused by increased blood flow absent the necessary vascular structure for proper venous return.

While V.A.C.® therapy has been highly successful in the promotion of wound closure, healing many wounds previously thought largely untreatable, some difficulty remains. Because the very nature of V.A.C.® therapy dictates an atmospherically sealed wound site, the therapy must often be performed to the exclusion of other beneficial, and therefore desirable, wound treatment modalities. One of these hitherto excluded modalities is the encouragement of cell growth by the provision of an in situ cell growth-enhancing matrix.

Additional difficulty remains in the frequent changing of the wound dressing. As the wound closes, binding of cellular tissue to the wound dressing may occur. Use of traditional V.A.C.® therapy necessitates regular changing of the dressing. Dressing changes can result in some tissue damage at the wound site if cellular tissue has grown excessively into the dressing.

U.S. Pat. No. 7,070,584, issued Jul. 4, 2006, discloses using a fused-fibrous ceramic, a bioabsorbable polymer or cell growth enhancing matrix or scaffolding in a V.A.C.® environment.

Accordingly, an object of the embodiments disclosed herein is to provide a wound dressing that effectively serves as a wound dressing for placement directly on to the wound and which provides keratin to the wound to promote healing.

A further object of the embodiments disclosed herein is to provide a wound dressing for placement into the wound that manages exudates, is bioabsorbable and reduces oxidative stress.

Another object of the embodiments disclosed herein is to provide an improved wound dressing for vacuum induced healing therapy, which overcomes the problems and limitations of the prior art.

An additional object of the embodiments disclosed herein is to allow for controlled application of growth factors or other healing factors, which could be embedded in the dressing or introduced into the dressing through a port or other connector fitting.

Still another object of the embodiments disclosed herein is to provide a fully and/or partially bioabsorbable wound dressing that minimizes disruption of the wound site during dressing changes.

A yet further object of the embodiments disclosed herein is to provide such a dressing that is economical and disposable, but also safe for general patient use.

### SUMMARY

In accordance with the foregoing objects, the present disclosure generally comprises a porous keratin construct for insertion substantially into the wound site. The porous keratin construct may be placed directly in the wound and optionally maintained in the wound through the use of, for example, a bandage, or may be used in conjunction with, for example, vacuum assisted closure as described in greater detail below.

In a first embodiment, the pad is a foamed solidified keratin protein material. The keratin protein is preferably S-sulfonated protein, oxidized keratin protein or reduced keratin protein. The keratin protein may also be keratin protein fractions, such as intermediate filament keratin protein, high-sulfur keratin protein or high-glycine-high-tryosine keratin protein. The keratin protein or protein fractions may be intact or hydrolysed.

In another embodiment, the pad comprises a conventional foam pad, such as a foam pad made of polyurethane or polyvinylalcohol, and a layer of porous keratin protein on the foam pad adjacent the wound, such that upon removal of the pad during dressing changes, the keratin protein is either left behind or has already bioabsorbed into the wound, leaving the wound site undisturbed. The porous keratin protein layer may be S-sulfonated protein, oxidized keratin protein or reduced keratin protein. The keratin protein adjacent the wound may also be a keratin protein fraction, such as intermediate filament keratin protein, high-sulfur keratin protein or high-glycine-high-tryosine keratin protein. The keratin protein or protein fraction may also be intact or hydrolysed.

In still another embodiment, either pad as described above is used as part of an assembly for vacuum assisted closure. In addition to the pad, the assembly may include a wound drape for enclosing the porous keratin construct or keratin construct and synthetic foam construct at the wound site. The keratin construct (with or without synthetic foam), comprised of a foamed solidified material having relatively few open cells in contact with the areas upon which cell growth is to be encouraged so as to avoid unwanted adhesions but having sufficiently numerous open cells so that drainage and vacuum assisted therapy may continue unimpaired, may be placed in the wound and encapsulated by the wound drape. Utilization of keratin in the pad enables the pad to remain in place during the healing process. As cell growth continues, the keratin material is absorbed, and there is no need to remove the pad. The assembly may also include a vacuum source for application of negative pressure to the area under the wound drape and promotion of fluid drainage. The wound drape forms an airtight seal over the wound site to prevent vacuum leakage.

Spaces in the porous keratin material create small volume areas that provide an excellent environment to enhance cell growth, and thus further the process envisioned by the healing process. Accordingly, cell growth enhancement therapy may be conveniently combined with existing vacuum assisted therapies, without loss of performance and without inconvenience or overly increased cost.

In still another embodiment, a method for treating wounds employing the construct described above is disclosed. The keratin construct may be placed in a wound and subsequently encapsulated by a wound drape. The wound drape may be placed in fluid communication with a vacuum source, and negative pressure may be applied to the area encapsulated by the wound drape.

The type of wound which may be treated by the above described embodiments is not limited and may include, for example, soft tissue wounds or bone defects.

Finally, many other features, objects and advantages of the present disclosure will be apparent to those of ordinary skill in the relevant arts, especially in light of the foregoing discussions and the following drawing and exemplary detailed description.

### BRIEF DESCRIPTION OF THE DRAWINGS

These and other features and advantages of the disclosure will now be described with reference to the drawings of certain preferred embodiments, which are intended to illustrate and not to limit the disclosure, and wherein like reference numbers refer to like components, and in which:

FIG. 1 shows, in partially cut away perspective view, a first embodiment of the present disclosure as applied to a mammalian wound site wherein a porous keratin pad is used in a vacuum assisted wound care environment;

FIG. 2 shows, in partially cut away perspective view, a second embodiment of the present disclosure as applied to a mammalian wound site wherein the porous keratin layer is used with a conventional foam pad in a vacuum assisted wound care environment.

### DETAILED DESCRIPTION

Although those of ordinary skill in the art will readily recognize many alternative embodiments, especially in light of the illustrations provided herein, this detailed description is exemplary of the preferred embodiment of the present disclosure, the scope of which is limited only by the claims that may be drawn hereto.

The present disclosure is directed to a biocompatible wound dressing which may be used by, for example, maintaining the wound dressing directly in the wound or in conjunction with negative pressure or vacuum assisted wound therapy. The term “wound” as used herein, while not limited, may include burns, incisional wounds, excisional wounds, ulcers, traumatic wounds, bone defects and chronic open wounds. As used herein, the term “construct,” while not limited, may include foams, screens, pads and blocks. The term “conventional pad,” while not limited, may include polyurethane (PU) or polyvinylalcohol (PVA) foam pads commonly used with vacuum assisted therapy.

In a first embodiment, a porous keratin construct is used in wound healing.

Keratin is a family of proteins characterized by a high degree of the amino acid cystine, which imparts a high degree of crosslinking to keratin proteins through disulfide links. Keratin proteins are present in a wide range of biological tissue, performing a structural role in skin, hair and other materials. Keratins extracted from hair have been shown to be a valuable component in wound dressings. Specifically, keratins have been found to be necessary for the re-epithelization phase of the wound healing process. Accordingly, a keratin construct used in negative pressure therapy will further promote wound healing and absorb into the wound, thus reducing the occurrence of traumatizing wounds when changing dressings or discontinuing use of negative pressure therapy.

The keratin protein of the present disclosure may be chemically modified. One such process involves chemically modifying keratin to form S-sulfonated keratin as described in U.S. Pat. No. 7,148,327, issued Dec. 12, 2006, incorporated herein by reference.

In one aspect, the keratin used in this disclosure is S-sulfonated keratin protein. S-sulfonated keratin refers to keratin protein that undergoes a process wherein the disulfide bonds between cystine amino acid in keratin protein are reversibly modified to create polar functional groups that allow for controlled re-introduction of the natural disulfide crosslinks originally present in the keratin protein. S-sulfonated keratins have cysteine/cystine present predominantly in the form of S-sulfocysteine. This highly polar group imparts a degree of solubility to proteins. Whilst being stable in solution, the S-sulfo group is a liable cysteine derivative, highly reactive towards thiols, such as cysteine, and other reducing agents. Reaction with reducing agents leads to conversion of the S-sulfo cysteine group back to cystine. S-sulfo cysteine is chemically different from cysteic acid, although both groups contain the SO₃⁻ group. Cysteic acid is produced irreversibly by the oxidation of cysteine or cystine and once formed cannot form disulfide crosslinks back to cysteine. S-sulfocysteine is reactive towards cysteine and readily forms disulfide crosslinks In the case of S-sulfonated keratin protein, the conversion of the S-sulfonate form to the crosslinked disulfide form may be accomplished through application of reducing conditions, for example, by applying a thiol. S-sulfonated keratin protein may be prepared by a variety of methods, including those described in U.S. Pat. No. 7,148,327, issued Dec. 12, 2006, incorporated herein by reference.

The mechanism for modifying the cystine disulfide bond to cysteine S-sulfonate is summarized as follows, wherein K is keratin:

K-S-S-K→2K-S-SO₃⁻

The mechanism for reforming the crosslinks may be summarized as follows, wherein K is keratin and R is a reducing agent:

K-S-SO₃⁻+R-S⁻→K-S-S-R+SO₃²⁻

K-S-S-R+R-S⁻→K-S-+R-S-S-R

K-S-SO₃⁻+R-S⁻→K-S-S-K+SO₃²⁻

The keratin protein may be a keratin protein fraction. Keratin protein fractions are distinct groups from within the keratin protein family, and include intermediate filament proteins, high sulfur proteins and high glycine-tyrosine proteins.

Intermediate filament proteins are described in detail by Orwin et al. (Structure and Biochemistry of Mammalian Hard Keratin, Electron Microscopy Reviews, 4, 47, 1991) and also referred to as low sulfur proteins by Gillespie (Biochemistry and physiology of the skin, vol. 1, Ed. Goldsmith Oxford University Press, London, 1983, pp. 475-510). Key characteristics of intermediate filament protein family are molecular weight in the range 40-60 kD and a cysteine content (measured as half cystine) of around 4%.

The high sulfur protein family is also well described by Orwin and Gillespie in the same publications reference above. This protein family has a large degree of heterogeity, but can be characterized as having a molecular weight in the range 10-30 kD and a cysteine content of greater than 10%. A subset of this family is the ultrahigh sulfur proteins, which can have a cysteine content of up to 34%.

The high glycine-tryosine protein family is also well described by Orwin and Gillespie in the same publications referenced above. This family is also referred to as the high tyrosine proteins and has characteristics of a molecular weight less than 10 kD, a tyrosine content typically greater than 10% and a glycine content typically greater than 20%.

For the purpose of this disclosure, a “keratin protein fraction” is a purified form of keratin that contains predominantly, although not entirely, one distinct protein group as described above.

The keratin protein or protein fraction may also be intact. The term intact refers to proteins that have not been significantly hydrolysed, with hydrolysis being defined as the cleavage of bonds through the addition of water. Gillespie considers intact to refer to proteins in the keratinized polymeric state and further refers to polypeptide subunits which complex to form intact keratin in wool and hair. For purposes of this disclosure, intact refers to the polypeptide subunits described in Gillespie. These are equivalent to the keratin proteins in their native form without the disulfide crosslinks formed through the process of keratinization.

Intact keratin proteins and keratin protein fractions are discussed in greater detail in co-pending, co-owned U.S. patent application Ser. No. 10/583,445, filed Jun. 19, 2006 and of which the entire application is hereby incorporated by reference.

The keratin may also be oxidized keratin. Oxidized keratins are produced as a result of exposing insoluble keratins to oxidizing agents, resulting in the conversion of cystine to cysteic acid and the keratin being converted to a soluble form. As a result of this, oxidized keratins are suitable for use in wound healing as disclosed herein.

The keratin may also be reduced keratin. Reduced keratins are produced as a result of exposing insoluble keratins to reducing agents, such as thiols, phosphines or other similar reducing agents. This converts the cystine present to cysteine or an alternative derivative, cleaving the crosslinks and converting the insoluble keratin into a soluble form. In this form, reduced keratins are soluble and suitable for use in wound healing as described herein.

In yet another alternate embodiment of the present disclosure, a conventional foam pad (e.g., a polyurethane foam or a polyvinylalcohol foam) further comprises a porous keratin protein growth-enhancing matrix layer facing towards a wound site. In this configuration, removal of the basic foam pad during dressing changes enables at least part of the porous keratin protein material to be left in the wound, thus leaving the wound site undisturbed. Furthermore, because the keratin is or comprises a material that is both bioabsorable and capable of promoting wound healing, the porous keratin further enhances negative pressure wound therapy when used for that purpose.

As with the previous embodiments, keratin protein may be S-sulfonated keratin protein, reduced keratin protein or oxidized keratin protein. The keratin protein may be a keratin protein fraction such as intermediate filament keratin protein, high sulfur keratin protein and high glycine-tyrosine keratin protein. The keratin protein or keratin protein fraction may be hydrolysed or intact.

Methods of making the porous keratin construct and keratin layer described above are set forth in commonly-owned, co-pending U.S. application Ser. No. 12/000,292, filed Dec. 11, 2007, the entirety of which is hereby incorporated by reference.

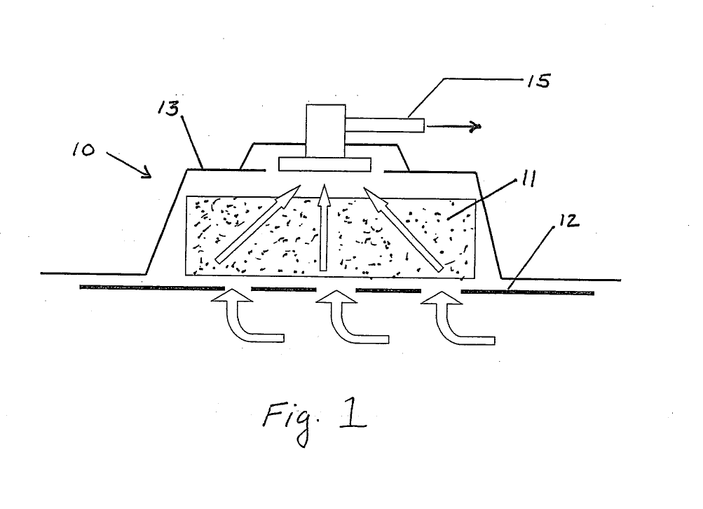

Referring now to the figures, a construct as described above and used in conjunction with known negative pressure therapy is shown in FIG. 1. Assemblies for use in negative pressure therapy generally comprise a porous keratin construct 11 for insertion substantially into the wound site 12, a wound drape 13 forming a sealing enclosure over the construct 11 at the wound site 12 and a vacuum source. According to one embodiment of the disclosure, the wound site is a soft tissue wound bed or a bone defect. The porous construct 11 may be made of or substantially comprise a solid, porous keratin protein. The porous keratin protein may be keratin protein fractions, intact and/or hydrolysed as discussed in greater detail above. In an alternate aspect of the embodiment, the porous construct 11 may be comprised of multiple, distinct layers of porous keratin. The layers may be separated from one another upon removal of the construct 11 from the wound so as to leave behind some layers.

After insertion of the keratin construct 11 into the wound site 12 and sealing with the wound drape 13, the wound drape 13 may be placed in fluid communication with a vacuum source and a negative pressure may be applied to the area encapsulated by the wound drape 13. Negative pressure is applied for promotion of fluid drainage in accordance with conventional procedures. The wound drape 13 may be placed in fluid communication, via a plastic or like material hose 15, with a vacuum source, which may comprise a canister safely placed under vacuum through fluid communication, via an interposed hydrophobic membrane filter, with a vacuum pump. The wound drape 13, which preferably may comprise an elastomeric material at least peripherally covered with a pressure sensitive, acrylic adhesive for sealing application over the wound site 12, is air tight so as to allow for negative pressure in the area enclosed by the wound drape 13. In one aspect, the construct 11 may also include perforations to reduce any pressure drop or impedance to exudate flow.

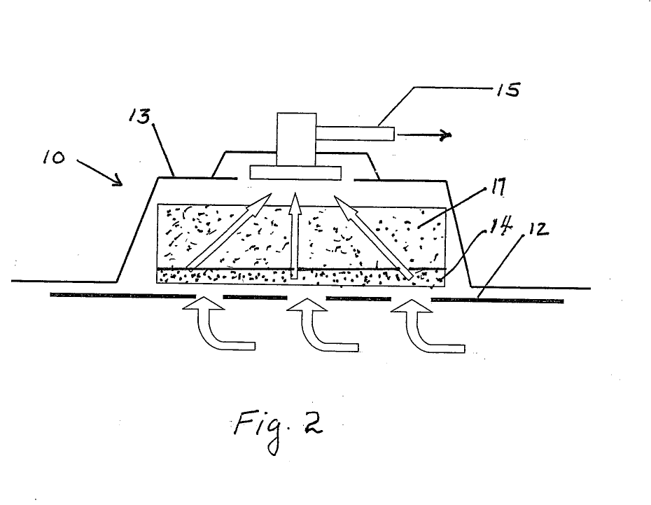

According to another embodiment of the instant disclosure and as illustrated in FIG. 2, a conventional foam pad 17 is modified to include a keratin layer 14, whereby a desired porous cell growth-enhancing construct that may be directed into and about the wound site 12 is provided. The keratin layer 14 may be, keratin protein fractions, hydrolysed and/or intact as described in greater detail above. The conventional pad 17 may be comprised of several distinct layers of conventional foam pads stacked on top of one another. Similarly, the keratin layer 14 may be comprised of several distinct layers of keratin layers stacked on top of one another.

After insertion of the foam pad 17 and keratin layer 14 into the wound site 12 and sealing with the wound drape 13, the wound drape 13 is placed in fluid communication with a vacuum source for promotion of fluid drainage in accordance with known procedures. The porous keratin layer 14 may cover the entire surface of the foam pad or only a portion thereof to suit specific wound care needs.

### EXAMPLE I

S-sulfonated keratin protein is formed into a porous pad. The general principles of known vacuum assisted wound therapy are followed with the pad in contact with the wound. During the expected duty cycle of the pad, the pad is partially or totally absorbed by the growing cells, so that there is less need to replace the pad and disturb the wound site.

### EXAMPLE II

A conventional foam pad used in vacuum assisted wound therapy is selected. A S-sulfonated keratin protein growth-enhancing porous layer is applied to a portion of the bottom thereof intended to face a wound site. The general principles of vacuum assisted wound therapy are followed, with the keratin layer containing pad substituted for a conventional pad. During the expected duty cycle of the pad, the keratin layer is absorbed by the growing cells, so that when the basic foam pad is removed, the keratin layer has been partially or totally absorbed, and the growing cells are not disturbed.

### EXAMPLE III

A porous solid pad formed of S-sulfonated keratin protein is selected. The pad is placed directly in a wound. The pad is secured on the wound by use of bandage or other securable means. During the expected duty cycle of the pad, the pad is absorbed by the growing cells, so that there is no need to replace the pad and disturb the wound site.

### EXAMPLE IV

A polymer foam or other conventional foam pad is selected. A solid porous S-sulfonated keratin protein growth-enhancing layer is applied to a portion of the bottom thereof intended to face a wound site. The composite pad is secured on the wound by use of bandage. During the expected duty cycle of the pad, the keratin layer is absorbed by the growing cells, so that when the pad is removed, the layer had been absorbed, and the growing cells are not disturbed.

### EXAMPLE V

### In Vitro Performance of Keratin Constructs

Using a bench top simulation rig, it was established that fluid could be drawn, at typical flow rates which prevail in highly exuding wounds, through a porous keratin construct or multiple layers of such constructs placed between a conventional polyurethane dressing and a wound surface without causing excessive pressure drop across the construct(s). Thus, it was demonstrated that said construct or constructs could be used adjacent to the polyurethane construct when administering negative pressure wound therapy without excessive loss of vacuum at the wound surface.

Further, when simulated wound fluid (Trypsin) was drawn through the porous keratin construct, it caused the construct to biodegrade, as is expected from experience with such constructs in wounds, and this reduced the pressure drop across the construct. This demonstrated that the biodegradation of the construct, which would be expected to occur in vivo, does not cause the construct to create an excessive pressure drop or loss of vacuum at the wound surface.

Still further, when simulated wound fluid (Trypsin) was drawn through multiple porous keratin constructs, the lowest construct (i.e., in direct contact with the wound upon first application) was observed to biodegrade first and there was a significant period of time when the lowest construct biodegraded but the upper porous keratin construct remained intact. This demonstrated that by using multiple porous keratin constructs in the wound bed under the conventional polyurethane construct, the benefits of a bioresorbable construct can be obtained whilst the upper construct remains intact and provides an interface to the conventional polyurethane construct and would prevent any tissue in-growth into the conventional polyurethane construct.

### EXAMPLE VI

### In Vivo Performance of Keratin Constructs

A clinical evaluation was performed on the use of a keratin construct as an adjunct to negative pressure wound therapy. In a series of cases of wound patients who would ordinarily receive negative pressure therapy, negative pressure wound therapy was administered using standard commercially available equipment involving a polyurethane foam and a vacuum pump typically set to 125-150 mmHg continuous negative pressure. In each case, pain at dressing change was evaluated prior to study commencement and again at each dressing change. Pain at dressing change typically occurs due to disruption of healing tissue as a result of in-growth into the polyurethane foam.

On commencement of the evaluation, keratin constructs were perforated with multiple 5 mm off-set incisions and hydrated in saline for approximately 3 minutes. These constructs were then placed under the polyurethane foam (i.e. at the wound interface), and negative pressure therapy continued in the normal manner. Dressing changes occurred typically 3 times per week. In several cases pain at dressing change was rated as 10 out of 10 prior to the study. By the third dressing change this had reduced to 0 out of 10, indicating a substantial reduction in pain at dressing change as a result of the keratin construct interface. Visual examination of the polyurethane foam indicated substantially less tissue in-growth following use of the keratin construct. In addition, exudate flows were reported as normal.

While the foregoing description is exemplary of the preferred embodiment of the present disclosure, those of ordinary skill in the relevant arts will recognize the many variations, alterations, modifications, substitutions and the like are readily possible, especially in light of this description and the accompanying drawings. In any case, because the scope of the present disclosure is much broader than any particular embodiment, the foregoing detailed description should not be construed as a limitation of the scope of the present disclosure, which is limited only by the claims that are drawn hereto.

We claim:

1. A bone defect or soft tissue wound healing assembly comprising:
a first porous keratin protein construct for positioning in a bone defect site or soft tissue wound bed;
a wound drape for encapsulating a bone defect site or soft tissue wound bed and the first porous keratin protein construct positioned therein; and
a vacuum in fluid communication with the wound drape for applying negative pressure to an area encapsulated by the wound drape.

2. The assembly of claim 1, wherein the first porous keratin protein construct comprises a keratin protein selected from the group consisting of S-sulfonated keratin protein, oxidized keratin protein and reduced keratin protein.

3. The assembly of claim 2, wherein the keratin protein is a keratin protein fraction.

4. The assembly of claim 3, wherein the keratin protein fraction is selected from the group consisting of intermediate filament protein, high sulfur protein and high glycine-tyrosine protein.

5. The assembly of claim 4, wherein the keratin protein fraction is intact.

6. The assembly of claim 5, wherein the keratin protein fraction is hydrolysed.

7. The assembly of claim 1, further comprising one or more supplemental porous keratin protein constructs layered on top of the first porous keratin protein construct.

8. The assembly of claim 1, wherein the first porous keratin protein construct comprises perforations.

9. A bone defect or soft tissue wound healing assembly comprising:
a first porous keratin protein construct for positioning in a bone defect site or soft tissue wound bed, the first porous keratin protein construct comprising:
a first surface for contacting a bone defect site or soft tissue wound bed; and
a second surface opposite the first surface;
a first synthetic foam construct positioned on the second surface of the first porous keratin protein construct;
a wound drape for encapsulating a bone defect site or soft tissue wound bed, the first porous keratin protein construct and the synthetic foam construct; and
a vacuum in fluid communication with the wound drape for applying negative pressure to an area encapsulated by the wound drape.

10. The assembly of claim 9, wherein the keratin protein construct comprises keratin protein selected from the group consisting of S-sulfonated keratin protein, oxidized keratin protein and reduced keratin protein.

11. The assembly of claim 10, wherein the keratin protein is a keratin protein fraction.

12. The assembly of claim 11, wherein the keratin protein fraction is selected from the group consisting of intermediate filament protein, high sulfur protein and high glycine-tyrosine protein.

13. The assembly of claim 12, wherein the keratin protein fraction is intact.

14. The assembly of claim 13, wherein the keratin protein fraction is hydrolysed.

15. The assembly of claim 9 further comprising one or more supplemental porous keratin protein constructs positioned between the first porous keratin protein construct and the first synthetic foam construct.

16. The assembly of claim 9, further comprising one or more supplemental synthetic foam construct positioned on top of the first synthetic foam construct.

17. The assembly of claim 9, wherein the first porous keratin protein construct comprises perforations.

18. A method for treating bone defects or soft tissue wounds comprising:
(1) positioning a porous keratin protein construct in a soft tissue wound bed or bone defect site;
(2) encapsulating the porous keratin protein construct and soft tissue wound bed or bone defect site with a wound drape to create an encapsulated area; and
(3) applying negative pressure to the encapsulated area;

19. The method of claim 18, wherein the method further comprises between steps (1) and (2), positioning a synthetic foam construct on the porous keratin protein construct.

20. The method of claim 18, wherein the porous keratin protein construct comprises a plurality of porous keratin protein constructs stacked on top of one another.

21. The method of claim 19, wherein the porous keratin protein construct comprises a plurality of porous keratin protein constructs stacked on top of one another.

22. The method of claim 19, wherein the synthetic foam construct comprises a plurality of synthetic foam constructs stacked on top of one another.

23. The method of claim 18, wherein the porous keratin protein construct comprises keratin protein selected from the group consisting of S-sulfonated keratin protein, oxidized keratin protein, and reduced keratin protein.

24. The method of claim 23, wherein the keratin protein is a keratin protein fraction.

25. The method of claim 24, wherein the keratin protein fraction is selected from the group consisting of intermediate filament protein, high sulfur protein, and high glycine-tyrosine protein.

26. The method of claim 25, wherein the keratin protein fraction is intact.

27. The method of claim 25, wherein the keratin protein is hydrolysed.

28. The method of claim 18, wherein the porous keratin protein construct comprises perforations.
